(19) United States
(12) Patent Application Publication
(10) Pub. No.: US 2016/0024556 A1
(43) Pub. Date: Jan. 28, 2016

(54) ENRICHMENT AND NEXT GENERATION SEQUENCING OF TOTAL NUCLEIC ACID COMPRISING BOTH GENOMIC DNA AND cDNA

(71) Applicant: ELIM BIOPHARMACEUTICALS, INC., Hayward, CA (US)

(72) Inventor: Yilin ZHANG, Hayward, CA (US)

(21) Appl. No.: 14/774,674

(22) PCT Filed: Mar. 10, 2014
(86) PCT No.: PCT/US2014/022566

§ 371 Date: Sep. 10, 2015

Related U.S. Application Data
(60) Provisional application No. 61/776,666, filed on Mar. 11, 2013.

(51) Int. Cl. C12Q 1/68; C12N 15/10

(52) U.S. Cl. CPC C12Q 1/6806; C12N 15/1093; C12Q 1/6874

(57) ABSTRACT

The present invention relates to methods of enriching and sequencing a mixture of nucleic acids comprising genomic DNA sequences and cDNA sequences obtained from the test sample.

### CROSS-REFERENCE TO RELATED APPLICATIONS

This Application claims the benefit of U.S. Provisional Application No. 61/776,666, filed on Mar. 11, 2013, entitled ENRICHMENT AND NEXT GENERATION SEQUENCING OF TOTAL NUCLEIC ACID, which is hereby incorporated by reference in its entity for all purposes.

### TECHNICAL FIELD

The present invention relates to next generation sequencing and disease diagnosis such as cancer diagnosis by analyzing a mixture of nucleic acids.

### BACKGROUND

Nucleic acid sequence analyses tools are fundamental for the identification of gene alterations, which in turn are useful for diagnosing genetic diseases, predicting responsiveness to drug treatments, and analyzing pharmacogenomics of drugs. One example is cancer diagnostics. Genetic variations that lead to cancer include single nucleotide variations (SNV), insertions and deletions (Indel), copy number variations (CNV), and translocations, etc. Because the analyses frequently involve the determination of rare genetic alterations in a limited amount of sample, sensitivity has been a big challenge. This is particularly true when analyzing somatic mutations in a tissue sample (such as a cancer sample), which frequently contains normal cells mixed with cells harboring the mutation.

Next generation sequencing (NGS) is a powerful tool for molecular profiling and characterization of different types of genetic variations associated with diseases such as cancer. The human genomic DNA is complex and has many repetitive sequences. This presents additional challenges for sequence analyses. First, nucleic acids of interest may be significantly under-represented among the mixture of nucleic acids. Second, the cost of analyzing the complex DNA sample can be prohibitively expensive, particularly in the context of analyzing genomic DNA and detecting multiple genetic mutations. While many next generation sequencing methods have been developed, there remains a need for sensitive, accurate, and efficient method for nucleic acid preparation and sequencing analyses.

The content of all references cited herein are incorporated by reference in their entirety.

### BRIEF SUMMARY OF THE INVENTION

The present application in one aspect provides a method of obtaining an enriched population of nucleic acids of interest from a test sample (such as a test human sample), comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; and (c) separating nucleic acids hybridized to said probes from those not hybridized; thereby obtaining an enriched population of nucleic acids of interest. In some embodiments, the mixture of nucleic acids is obtained by mixing a genomic DNA library and a cDNA library generated from the test sample. In some embodiments, the mixture of nucleic acids is obtained by (i) reverse transcribing the RNA in the test sample into cDNA and (ii) generating a DNA library comprising genomic DNA sequences and cDNA sequences to provide a mixture of nucleic acids.

In some embodiments according to (or as applied to) any of the embodiments above, at least one of the probes is complementary to a nucleic acid of interest present in a genomic DNA sequence and a nucleic acid of interest present in a cDNA sequence.

In some embodiments according to (or as applied to) any of the embodiments above, the genomic DNA sequence and cDNA sequence are present in the mixture in a predetermined ratio.

In some embodiments according to (or as applied to) any of the embodiments above, the nucleic acids of interest comprise a plurality of exon sequences, a plurality of intron sequences, a plurality of intron-exon junctions, or a plurality of sequences in a non-coding region.

In some embodiments according to (or as applied to) any of the embodiments above, the set of probes comprises at least about 100 different probes.

In some embodiments according to (or as applied to) any of the embodiments above, the probes are in at least about 10× molar excess compared to complementary regions within the nucleic acid mixture.

In some embodiments according to (or as applied to) any of the embodiments above, the probes comprise sequences complementary to an oncogene, a tumor suppressor, a tyrosine kinase, a phosphatase, or a vascular gene.

In some embodiments according to (or as applied to) any of the embodiments above, the probes are attached to a solid support prior to or after being in contact with the mixture of nucleic acids. In some embodiments, the method further comprises eluting the probes and nucleic acids of interest hybridized to the probes from the solid support.

In some embodiments according to (or as applied to) any of the embodiments above, further comprising amplifying said nucleic acids of interest.

In some embodiments according to (or as applied to) any of the embodiments above, further comprising analyzing the enriched nucleic acids, such as sequencing the enriched nucleic acids of interest.

In another aspect, there is provided a method of characterizing nucleic acids in a test sample, comprising: (a) providing a mixture of nucleic acids comprising genomic DNA sequences and cDNA sequences obtained from the test sample; and (b) simultaneously sequencing the genomic DNA sequences and cDNA sequences in the mixture. In some embodiments, the mixture of nucleic acids is obtained by mixing a genomic DNA library and a cDNA library generated from the test sample. In some embodiments, the mixture of nucleic acids is obtained by (i) reverse transcribing the RNA in the test sample into cDNA and (ii) generating a DNA library comprising genomic DNA sequences and cDNA sequences to provide a mixture of nucleic acids.

In some embodiments according to any of the embodiments above, the characterization comprises determination of variations in the genomic DNA sequence in the test sample, which include, but are not limited to, chromosomal rearrangement, single nucleotide variation (SNV), or copy number variation (CNV). In some embodiments, the chromosomal rearrangement comprises deletion, insertion, and translocation of DNA sequences.

In some embodiments according to any of the embodiments above, the characterization comprises determination of variations in the RNA transcripts in the test sample, which include, but are not limited to, deletion, insertion, translocation, SNV, or differential gene expression.

In some embodiments according to any of the embodiments above, wherein the method comprises enriching the nucleic acid mixture for nucleic acids of interest prior to the sequencing step. In some embodiments, the enrichment comprises: (a) contacting the mixture of nucleic acids with a set of probes under a condition sufficient for hybridization of said nucleic acids to said probes, wherein said probes are complementary to nucleic acids of interest present in said nucleic acid mixture; and (b) separating nucleic acids that are hybridized to said probes from those not hybridized; thereby obtaining an enriched population of nucleic acids of interest.

In some embodiments according to any of the embodiments above, the method further comprises adding to the enriched population of nucleic acids the initial mixture of nucleic acids prior to the sequencing step. In an alternative embodiment, the method further comprises adding to the enriched population of nucleic acids genomic DNA sequences prior to the sequencing step. In yet another alternative embodiment, the method further comprises adding to the enriched population of nucleic acids cDNA sequences prior to the sequencing step.

In some embodiments according to any of the embodiments above, the nucleic acid mixture further comprise genomic DNA sequences from a control sample, for example a control sample from the same or a different individual.

In some embodiments according to any of the embodiments above, the nucleic acid mixture further comprises cDNA sequences from a control sample, for example a control sample from the same or a different individual.

Also provided are kits and articles of manufacture suitable for any one of the methods described herein.

Additional embodiments, features, and advantages of the invention will be apparent from the following detailed description and through practice of the invention.

For the sake of brevity, the disclosures of the publications cited in this specification, including patents, are herein incorporated by reference.

### DETAILED DESCRIPTION OF THE INVENTION

The present invention provides nucleic acid preparation and enrichment methods that allows simultaneous analysis and sequencing of genomic DNA and RNA (cDNA) derived from the same test sample (for example a test sample from a single individual). The simultaneous analysis maximizes the utilization of rare and precious samples and simplifies nucleic acid manipulation and analyses in a clinical setting. The combined analyses of genomic DNA and RNA (cDNA) provide complementary information about the genome and the transcriptome in the test sample. This makes it possible to obtain a complete nucleic acid profile of the test sample that reflects both genomic variations and variations at the transcriptional level. In addition, information obtained by analyzing the genomic DNA and those obtained by analyzing the RNA (cDNA) may overlap with each other, thus allowing mutual validation and increasing confidence in nucleic acid analyses.

Thus, the present invention in one aspect provides a method of obtaining an enriched population of nucleic acids of interest from a mixture of nucleic acids comprising genomic DNA sequences and cDNA sequences obtained from the same test sample.

In another aspect, there is provided a method of characterizing (such as sequencing) nucleic acids in a mixture nucleic acids comprising genomic DNA sequences and cDNA sequences obtained from the same test sample.

Kits, compositions, and articles of manufacture useful for methods described herein are also provided.

### Definitions

The term “enrichment” refers to the process of increasing the relative abundance of particular nucleic acid sequences in a sample relative to the level of nucleic acid sequences as a whole initially present in said sample before enrichment. Thus the enrichment step provides a relative percentage or fractional increase, rather than directly increasing, for example, the absolute copy number of the nucleic acid sequences of interest. After the step of enrichment, the sample may be referred to as an enriched nucleic acid population.

As used herein, the “complexity” of a nucleic acid sample refers to the number of different unique sequences present in that sample. A sample is considered to have “reduced complexity” if it is less complex than the nucleic acid sample from which it is derived.

As used herein, “solid support” refers to a solid or semisolid material which has the property, either inherently or through attachment of some component conferring the property (e.g., an antibody, streptavidin, nucleic acid, or other binding ligands), of binding to a tag. Such binding may be direct or indirect. Examples of solid support include, but are not limited to, nitrocellulose and nylon membranes, agarose or cellulose based beads (e.g., Sepharose) and paramagnetic beads.

As used herein, the term “library” refers to a collection of nucleic acid sequences.

As used herein, the term “hybridize specifically” means that nucleic acids hybridize with a nucleic acid of complementary sequence. As used herein, a portion of a nucleic acid molecule may hybridize specifically with a complementary sequence on another nucleic acid molecule. That is, the entire length of a nucleic acid sequence does not necessarily need to hybridize for a portion of such sequence to be “hybridized specifically” to another molecule.

A “portion” or “region,” used interchangeably herein, of a nucleic acid or oligonucleotide is a contiguous sequence of 2 or more bases. In other embodiments, a region or portion is at least about any of 3, 5, 10, 15, 20, 25 contiguous nucleotides.

Sequence “mutation” or “variation” as used herein, refers to any sequence alteration in a sequence of interest in comparison to a reference sequence. A reference sequence can be a wild type sequence or a sequence to which one wishes to compare a sequence of interest. A mutation includes a single nucleotide change or alterations of more than one nucleotide in a sequence, due to mechanisms such as substitution, deletion or insertion.

A nucleic acid or primer is “complementary” to another nucleic acid when at least two contiguous bases of, e.g., a first nucleic acid or a primer, can combine in an antiparallel association or hybridize with at least a subsequence of a second nucleic acid to form a duplex. In some embodiments, complementarity between e.g., a primer and a target nucleic acid sequence, is not 100% perfect.

The term “nucleic acid of interest” used herein refers to a nucleic acid that is of interest to the investigator.

“Amplification,” as used herein, generally refers to the process of producing two or more copies of a desired sequence. “Nucleic acid” as used herein refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A nucleic acid may comprise modified nucleotides, such as methylated nucleotides and their analogs.

“Oligonucleotide,” as used herein, generally refers to short, generally single stranded, generally synthetic nucleic acids that are generally, but not necessarily, no more than about 200 nucleotides in length. The terms “oligonucleotide” and “nucleic acid” are not mutually exclusive. The description above for nucleic acids is equally and fully applicable to oligonucleotides.

“Fragmenting” a nucleic acid used herein refers to breaking the nucleic acids into different nucleic acid fragments. Fragmenting can be achieved, for example, by shearing or by enzymatic reactions.

A “primer” is generally a short single stranded nucleic acid, generally with a free 3′-OH group, that binds to a target of interest by hybridizing with a target sequence, and thereafter promotes polymerization of a nucleic acid complementary to the target.

“Hybridization” and “annealing” refer to a reaction in which one or more nucleic acids react to form a complex that is stabilized via hydrogen bonding between the bases of the nucleotide residues. The hydrogen bonding may occur by Watson-Crick base pairing, Hoogstein binding, or in any other sequence specific manner.

An “adaptor” used herein refers to an oligonucleotide that can be joined to a nucleic acid fragment.

The term “ligation” as used herein, with respect to two nucleic acids, such as an adaptor and a nucleic acid fragment, refers to the covalent attachment of two separate nucleic acids to produce a single larger nucleic acid with a contiguous backbone.

The term “3′” generally refers to a region or position in a nucleic acid or oligonucleotide that is downstream of another region or position in the same nucleic acid or oligonucleotide.

The term “5′” generally refers to a region or position in a nucleic acid or oligonucleotide that is upstream from another region or position in the same nucleic acid or oligonucleotide.

An “array” used herein includes arrangement of spatially or optically addressable regions bearing nucleic acids or other molecules. When the arrays are arrays of nucleic acids, the nucleic acids may be physically absorbed, chemically absorbed, or covalently attached to the arrays at any point or points along the nucleic acid chain.

As used herein, the term “single nucleotide variation,” or “SNV” for short, refers to change at a single nucleotide position in a genomic sequence relative to a wild type allele.

The term “copy number variation” or “CNV” for short, refers to change in gene copy number in a genomic sequence relative to a wild type genomic DNA.

The term “denaturing” as used herein refers to the separation of a nucleic acid duplex into two single strands.

It is understood that aspect and embodiments of the invention described herein include “consisting” and/or “consisting essentially of” aspects and embodiments.

As used herein, the singular form “a”, “an”, and “the” includes plural references unless indicated otherwise.

As is understood by one skilled in the art, reference to “about” a value or parameter herein includes (and describes) embodiments that are directed to that value or parameter per se. For example, description referring to “about X” includes description of “X”.

### Methods of the Present Invention

The present application in some embodiments provides a method of obtaining an enriched population of nucleic acids of interest from a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; and (b) separating nucleic acids hybridized to said probes from those not hybridized; thereby obtaining an enriched population of nucleic acids of interest.

In some embodiments, there is provided a method of obtaining an enriched population of nucleic acids of interest from a test sample, comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; and (c) separating nucleic acids hybridized to said probes from those not hybridized; thereby obtaining an enriched population of nucleic acids of interest.

In some embodiments, there is provided a method of obtaining an enriched population of nucleic acids of interest from a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; and (c) separating nucleic acids hybridized to said probes from those not hybridized; thereby obtaining an enriched population of nucleic acids of interest. In some embodiments, there is provided a method of obtaining an enriched population of nucleic acids of interest from a test sample, comprising: (a) mixing a genomic DNA library generated from the test sample and a cDNA library generated from the test sample at a predetermined ratio (for example at a ratio of about 10:1, 5:1, 2:1, 1:1, 1:2, 1:5, 1:10) to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; and (c) separating nucleic acids hybridized to said probes from those not hybridized; thereby obtaining an enriched population of nucleic acids of interest. In some embodiments, the cDNA library is prepared from total RNA in the test sample, which include, for example, mRNA, ribosomal RNA, nuclear RNA, cytoplasmic RNA, capped RNA, and small RNA. In some embodiments, the cDNA library is prepared from a processed RNA sample with ribosomal RNA removed. In some embodiments, the cDNA library is prepared from mRNA.

In some embodiments, there is provided a method of obtaining an enriched population of nucleic acids of interest from a test sample, comprising: (a) reverse transcribing the RNA in the test sample into cDNA; (b) generating a DNA library comprising genomic DNA sequences and cDNA sequences to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; and (c) separating nucleic acids hybridized to said probes from those not hybridized; thereby obtaining an enriched population of nucleic acids of interest. In some embodiments, the reverse transcription is carried out with total RNA in the test sample, which include, for example, mRNA, ribosomal RNA, nuclear RNA, cytoplasmic RNA, capped RNA, and small RNA. In some embodiments, the reverse transcription is carried out with a processed RNA sample with ribosomal RNA removed. In some embodiments, the reverse transcription is carried out with mRNA.

In some embodiments, the method further comprises analyzing (such as sequencing) the enriched nucleic acids of interest. In some embodiments, the method further comprises amplifying the nucleic acids of interest prior to the analyses.

In another aspect, the present application provides a method of characterizing nucleic acids in a test sample, comprising simultaneously sequencing genomic DNA sequences and cDNA sequences in a nucleic acid mixture comprising genomic DNA sequences and cDNA sequences obtained from the test sample. In some embodiments, there is provided a method of characterizing nucleic acids in a test sample, comprising: (a) providing a mixture of nucleic acids comprising genomic DNA sequences and cDNA sequences obtained from the test sample; and (b) sequencing the genomic DNA sequences and cDNA sequences in the mixture. In some embodiments, there is provided a method of characterizing nucleic acids in a test sample, comprising: (a) mixing a genomic DNA library generated from the test sample and a cDNA library generated from the test sample to provide a mixture of nucleic acids; and (b) sequencing the genomic DNA sequences and cDNA sequences in the mixture. In some embodiments, there is provided a method of characterizing nucleic acids in a test sample, comprising: (a) mixing a genomic DNA library generated from the test sample and a cDNA library generated from the test sample at a predetermined ratio (for example at a ratio of about 10:1, 5:1, 2:1, 1:1, 1:2, 1:5, 1:10) to provide a mixture of nucleic acids; and (b) sequencing the genomic DNA sequences and cDNA sequences in the mixture. In some embodiments, the cDNA library is prepared from total RNA in the test sample, which include, for example, mRNA, ribosomal RNA, nuclear RNA, cytoplasmic RNA, capped RNA, and small RNA. In some embodiments, the cDNA library is prepared from a processed RNA sample with ribosomal RNA removed. In some embodiments, the cDNA library is prepared from mRNA.

In some embodiments, there is provided a method of characterizing nucleic acids in a test sample, comprising: (a) reverse transcribing the RNA in the test sample into cDNA; (b) generating a DNA library comprising genomic DNA sequences and cDNA sequences to provide a mixture of nucleic acids; and (c) sequencing the genomic DNA sequences and cDNA sequences in the mixture. In some embodiments, reverse transcription is carried out with total RNA in the test sample, which include, for example, mRNA, ribosomal RNA, nuclear RNA, cytoplasmic RNA, capped RNA, and small RNA. In some embodiments, reverse transcription is carried out with a processed RNA sample with ribosomal RNA removed. In some embodiments, reverse transcription is carried out with mRNA.

In some embodiments, the mixture of nucleic acids is subjected to an enrichment step prior to the analyses. Thus, for example, in some embodiments, there is provided a method of characterizing nucleic acids in a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; (b) separating nucleic acids hybridized to said probes from those not hybridized; thereby obtaining an enriched population of nucleic acids of interest; and (c) sequencing the nucleic acid of interest.

In some embodiments, there is provided a method of characterizing nucleic acids in a test sample, comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; and (d) sequencing the nucleic acids of interest. In some embodiments, there is provided a method of characterizing nucleic acids in a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; and (d) sequencing the nucleic acids of interest. In some embodiments, the genomic DNA library and the cDNA library are mixed at a predetermined ratio (for example at a ratio of about 10:1, 5:1, 2:1, 1:1, 1:2, 1:5, 1:10).

In some embodiments, there is provided a method of characterizing nucleic acids in a test sample, comprising: (a) reverse transcribing the RNA in the test sample into cDNA; (b) generating a DNA library comprising genomic DNA sequences and cDNA sequences to provide a mixture of nucleic acids; (c) contacting the mixture of nucleic acids with a set of probes under a condition sufficient for hybridization of said nucleic acids to said probes, wherein said probes are complementary to nucleic acids of interest present in said mixture of nucleic acids; (d) separating nucleic acids hybridized to said probes from those not hybridized; thereby obtaining an enriched population of nucleic acids of interest; and (e) sequencing the nucleic acids of interest.

In some embodiments, there is provided a method of characterizing nucleic acids in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (d) mixing said enriched genomic DNA of interest and said enriched cDNA of interest to obtain a population of nucleic acids of interest; and (e) sequencing the nucleic acids of interest. In some embodiments, the enriched genomic DNA of interest and the enriched cDNA of interest are mixed at a predetermined ratio (for example at a ratio of about 10:1, 5:1, 2:1, 1:1, 1:2, 1:5, 1:10).

The methods described herein can be useful for any one of the nucleic acid analytical methods, including, but not limited to, obtaining a nucleic acid profile of the genome and/or transcriptome, sequencing a nucleic acid, determining the presence or absence of a variation in a nucleic acid, analyzing the polymorphism of the nucleic acid, analyzing copy number variation in the nucleic acids, analyzing gene expression level in the test sample, and the like.

Thus, for example, in some embodiments, there is provided a method of obtaining a nucleic acid profile of the genome and transcriptome in a test sample, comprising simultaneously sequencing genomic DNA sequences and cDNA sequences in a nucleic acid mixture comprising genomic DNA sequences and cDNA sequences obtained from the test sample. In some embodiments, there is provided a method of obtaining a nucleic acid profile of the genome and transcriptome in a test sample, comprising: (a) providing a mixture of nucleic acids comprising genomic DNA sequences and cDNA sequences obtained from the test sample; and (b) sequencing the genomic DNA sequences and cDNA sequences in the mixture. In some embodiments, there is provided a method of obtaining a nucleic acid profile of the genome and transcriptome in a test sample, comprising: (a) mixing a genomic DNA library generated from the test sample and a cDNA library generated from the test sample to provide a mixture of nucleic acids; and (b) sequencing the genomic DNA sequences and cDNA sequences in the mixture. In some embodiments, there is provided a method of obtaining a nucleic acid profile of the genome and transcriptome in a test sample, comprising: (a) mixing a genomic DNA library generated from the test sample and a cDNA library generated from the test sample at a predetermined ratio (for example at a ratio of about 10:1, 5:1, 2:1, 1:1, 1:2, 1:5, 1:10) to provide a mixture of nucleic acids; and (b) sequencing the genomic DNA sequences and cDNA sequences in the mixture. In some embodiments, the cDNA library is prepared from total RNA in the test sample, which include, for example, mRNA, ribosomal RNA, nuclear RNA, cytoplasmic RNA, capped RNA, and small RNA. In some embodiments, the cDNA library is prepared from a processed RNA sample with ribosomal RNA removed. In some embodiments, the cDNA library is prepared from mRNA.

In some embodiments, there is provided a method of obtaining a nucleic acid profile of the genome and transcriptome in a test sample, comprising: (a) reverse transcribing the RNA in the test sample into cDNA; (b) generating a DNA library comprising genomic DNA sequences and cDNA sequences to provide a mixture of nucleic acids; and (c) sequencing the genomic DNA sequences and cDNA sequences in the mixture. In some embodiments, reverse transcription is carried out with total RNA in the test sample, which include, for example, mRNA, ribosomal RNA, nuclear RNA, cytoplasmic RNA, capped RNA, and small RNA. In some embodiments, reverse transcription is carried out with a processed RNA sample with ribosomal RNA removed. In some embodiments, reverse transcription is carried out with mRNA.

In some embodiments, there is provided a method of obtaining a nucleic acid profile of genomic DNA and RNA of interest in a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; (b) separating nucleic acids hybridized to said probes from those not hybridized; thereby obtaining an enriched population of nucleic acids of interest; and (c) sequencing the nucleic acid of interest. In some embodiments, there is provided a method of obtaining a nucleic acid profile of genomic DNA and RNA of interest in a test sample, comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; and (d) sequencing the nucleic acids of interest. In some embodiments, there is provided a method of obtaining a nucleic acid profile of genomic DNA and RNA of interest in a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; and (d) sequencing the nucleic acids of interest. In some embodiments, the genomic DNA library and the cDNA library are mixed at a predetermined ratio (for example at a ratio of about 10:1, 5:1, 2:1, 1:1, 1:2, 1:5, 1:10).

In some embodiments, there is provided a method of obtaining a nucleic acid profile of genomic DNA and RNA of interest in a test sample, comprising: (a) reverse transcribing the RNA in the test sample into cDNA; (b) generating a DNA library comprising genomic DNA sequences and cDNA sequences to provide a mixture of nucleic acids; (c) contacting the mixture of nucleic acids with a set of probes under a condition sufficient for hybridization of said nucleic acids to said probes, wherein said probes are complementary to nucleic acids of interest present in said mixture of nucleic acids; (d) separating nucleic acids hybridized to said probes from those not hybridized; thereby obtaining an enriched population of nucleic acids of interest; and (e) sequencing the nucleic acids of interest.

In some embodiments, there is provided obtaining a nucleic acid profile of genomic DNA and RNA of interest in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (d) mixing said enriched genomic DNA of interest and said enriched cDNA of interest to obtain a population of nucleic acids of interest; and (e) sequencing the nucleic acids of interest. In some embodiments, the enriched genomic DNA of interest and the enriched cDNA of interest are mixed at a predetermined ratio (for example at a ratio of about 10:1, 5:1, 2:1, 1:1, 1:2, 1:5, 1:10).

In some embodiments, there is provided a method of simultaneously determining a genetic variation and variations in a RNA transcript in a test sample, comprising simultaneously sequencing genomic DNA sequences and cDNA sequences in a nucleic acid mixture comprising genomic DNA sequences and cDNA sequences obtained from the test sample. In some embodiments, there is provided a method of simultaneously determining a genetic variation and variations in a RNA transcript comprising: (a) providing a mixture of nucleic acids comprising genomic DNA sequences and cDNA sequences obtained from the test sample; and (b) sequencing the genomic DNA sequences and cDNA sequences in the mixture. In some embodiments, there is provided a method of simultaneously determining a genetic variation and variations in a RNA transcript, comprising: (a) mixing a genomic DNA library generated from the test sample and a cDNA library generated from the test sample to provide a mixture of nucleic acids; and (b) sequencing the genomic DNA sequences and cDNA sequences in the mixture. In some embodiments, there is provided a method of simultaneously determining a genetic variation and variations in a RNA transcript, comprising: (a) mixing a genomic DNA library generated from the test sample and a cDNA library generated from the test sample at a predetermined ratio (for example at a ratio of about 10:1, 5:1, 2:1, 1:1, 1:2, 1:5, 1:10) to provide a mixture of nucleic acids; and (b) sequencing the genomic DNA sequences and cDNA sequences in the mixture. In some embodiments, the cDNA library is prepared from total RNA in the test sample, which include, for example, mRNA, ribosomal RNA, nuclear RNA, cytoplasmic RNA, capped RNA, and small RNA. In some embodiments, the cDNA library is prepared from a processed RNA sample with ribosomal RNA removed. In some embodiments, the cDNA library is prepared from mRNA.

In some embodiments, there is provided a method of simultaneously determining a genetic variation and variations in a RNA transcript in a test sample, comprising: (a) reverse transcribing the RNA in the test sample into cDNA; (b) generating a DNA library comprising genomic DNA sequences and cDNA sequences to provide a mixture of nucleic acids; and (c) sequencing the genomic DNA sequences and cDNA sequences in the mixture. In some embodiments, reverse transcription is carried out with total RNA in the test sample, which include, for example, mRNA, ribosomal RNA, nuclear RNA, cytoplasmic RNA, capped RNA, and small RNA. In some embodiments, reverse transcription is carried out with a processed RNA sample with ribosomal RNA removed. In some embodiments, reverse transcription is carried out with mRNA.

In some embodiments, there is provided a method of simultaneously determining a genetic variation and variations in a RNA transcript in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (d) mixing said enriched genomic DNA of interest and said enriched cDNA of interest to obtain a population of nucleic acids of interest; and (e) sequencing the nucleic acids of interest. In some embodiments, the enriched genomic DNA of interest and the enriched cDNA of interest are mixed at a predetermined ratio (for example at a ratio of about 10:1, 5:1, 2:1, 1:1, 1:2, 1:5, 1:10).

In some embodiments, there is provided a method of simultaneously determining a genetic variation and variations in a RNA transcript of nucleic acids of interest in a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; (b) separating nucleic acids hybridized to said probes from those not hybridized; thereby obtaining an enriched population of nucleic acids of interest; and (c) sequencing the nucleic acid of interest. In some embodiments, there is provided a method of simultaneously determining a genetic variation and variations in a RNA transcript of nucleic acids of interest in a test sample, comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; and (d) sequencing the nucleic acids of interest. In some embodiments, there is provided a method of simultaneously determining a genetic variation and variations in a RNA transcript of nucleic acids of interest in a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; and (d) sequencing the nucleic acids of interest. In some embodiments, the genomic DNA library and the cDNA library are mixed at a predetermined ratio (for example at a ratio of about 10:1, 5:1, 2:1, 1:1, 1:2, 1:5, 1:10).

In some embodiments, there is provided a method of simultaneously determining a genetic variation and variations in a RNA transcript of nucleic acids of interest in a test sample, comprising: (a) reverse transcribing the RNA in the test sample into cDNA; (b) generating a DNA library comprising genomic DNA sequences and cDNA sequences to provide a mixture of nucleic acids; (c) contacting the mixture of nucleic acids with a set of probes under a condition sufficient for hybridization of said nucleic acids to said probes, wherein said probes are complementary to nucleic acids of interest present in said mixture of nucleic acids; (d) separating nucleic acids hybridized to said probes from those not hybridized; thereby obtaining an enriched population of nucleic acids of interest; and (e) sequencing the nucleic acids of interest.

The methods described herein can be useful for analyzing a nucleic acid sample from an individual, which can be useful for purposes that include, but are not limited to: 1) diagnosing a disease (such as cancer) in an individual, 2) assessing risk of developing a disease (such as cancer) in an individual, 3) determining responsiveness of an individual to a treatment regime (such as cancer treatment), 4) evaluating efficacy of a treatment (such as cancer treatment) on an individual, 5) determining continued treatment (such as cancer treatment) on an individual; and 6) predicting responsiveness of an individual to a treatment regime (such as cancer). In some embodiments, the methods are useful for genetic testing (such as prenatal screening). In some embodiments, the methods are useful for predicting pharmacokinetics of a drug in an individual.

The methods described herein are particularly useful in a personalized medicine setting, where the nucleic acid profile including information about genomic DNA and RNA of an individual is determined and used as a guide for devising a personalized treatment regime. The ability to obtain information on genomic DNA and RNA from the sample of the individual maximizes the use of the sample and makes the clinical testing simple and efficient.

In some embodiments, the mixture of nucleic acids from the test sample may further comprise control genomic DNA sequences and/or control cDNA sequences. These control sequences are separately indexed to facilitate data analyses and comparison. The control sequences may be derived from the same individual. For example, in some embodiments when the test sample is a tumor sample, the control sequences may be derived from a control sample from the normal tissue of the same individual. In some embodiments, the control sequences are derived from a control sample obtained from a different individual, such as an individual not diagnosed with a disease.

In some embodiments, a mixture of nucleic acids may be obtained by combining a nucleic acid mixture prior to enrichment and an enriched population of nucleic acids of interest at a predetermined ratio (for example at a ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000). This allows both broad sequencing (or analysis) at the genome-wide and/or transcriptome-wide level and deep sequencing of the nucleic acids of interest.

Thus, for example, In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; (b) separating nucleic acids hybridized to said probes from those not hybridized; thereby obtaining an enriched population of nucleic acids of interest; (c) adding to the enriched population of nucleic acids of interest the initial mixture of nucleic acids at a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000 for mixture:enriched), and (d) sequencing the genomic DNA sequences and cDNA sequences in the new mixture. In some embodiments, there is provided a method of characterizing nucleic acid (such as obtaining a nucleic acid profile of genomic DNA and RNA and/or simultaneously detecting genetic variations and variations in a RNA transcript): (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; (d) adding to the enriched population of nucleic acids of interest the initial mixture of nucleic acids at a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000 for mixture:enriched), and (e) sequencing the genomic DNA sequences and cDNA sequences in the new mixture. In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; (d) adding to the enriched population of nucleic acids of interest the initial mixture of nucleic acids at a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000 for mixture:enriched), and (e) sequencing the genomic DNA sequences and cDNA sequences in the new mixture. In some embodiments, the genomic DNA library and the cDNA library are mixed at a predetermined ratio (for example at a ratio of about 10:1, 5:1, 2:1, 1:1, 1:2, 1:5, 1:10).

In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) reverse transcribing the RNA in the test sample into cDNA; (b) generating a DNA library comprising genomic DNA sequences and cDNA sequences to provide a mixture of nucleic acids; (c) contacting the mixture of nucleic acids with a set of probes under a condition sufficient for hybridization of said nucleic acids to said probes, wherein said probes are complementary to nucleic acids of interest present in said mixture of nucleic acids; (d) separating nucleic acids hybridized to said probes from those not hybridized; thereby obtaining an enriched population of nucleic acids of interest; (e) adding to the enriched population of nucleic acids of interest the initial mixture of nucleic acids at a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000 for mixture:enriched), and (f) sequencing the genomic DNA sequences and cDNA sequences in the new mixture.

In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (e) mixing said enriched genomic DNA of interest and said enriched cDNA of interest to obtain a population of nucleic acids of interest; (f) adding to the population of nucleic acids of interest a mixture of the genomic DNA and cDNA libraries prior to the enrichments at a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000 for unenriched:enriched), and (f) sequencing the genomic DNA sequences and cDNA sequences in the new mixture.

In some embodiments, genomic DNA sequences obtained from the same test sample can be added to the enriched nucleic acid mixture. The addition of genomic DNA sequences allows, for example, both broad sequencing (or analyzing) at the genome-wide level and deep sequencing of the nucleic acids of interest. In some embodiments, the desired ratio of genomic DNA sequences to the nucleic acid mixture is about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000.

Thus, for example, In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; (b) separating nucleic acids hybridized to said probes from those not hybridized; thereby obtaining an enriched population of nucleic acids of interest; (c) adding to the enriched population of nucleic acids of interest genomic DNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of genomic DNA sequences and the enriched nucleic acid mixture, and (d) sequencing the genomic DNA sequences and cDNA sequences in the new mixture. In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; (d) adding to the enriched population of nucleic acids of interest genomic DNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of genomic DNA sequences and the enriched nucleic acid mixture, and (e) sequencing the genomic DNA sequences and cDNA sequences in the new mixture. In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; (d) adding to the enriched population of nucleic acids of interest genomic DNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of genomic DNA sequences the enriched nucleic acid mixture, and (e) sequencing the genomic DNA sequences and cDNA sequences in the new mixture. In some embodiments, the genomic DNA library and the cDNA library are mixed before the enrichment at a predetermined ratio (for example at a ratio of about 10:1, 5:1, 2:1, 1:1, 1:0, 0:1, 1:2, 1:5, 1:10).

In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) reverse transcribing the RNA in the test sample into cDNA; (b) generating a DNA library comprising genomic DNA sequences and cDNA sequences to provide a mixture of nucleic acids; (c) contacting the mixture of nucleic acids with a set of probes under a condition sufficient for hybridization of said nucleic acids to said probes, wherein said probes are complementary to nucleic acids of interest present in said mixture of nucleic acids; (d) separating nucleic acids hybridized to said probes from those not hybridized; thereby obtaining an enriched population of nucleic acids of interest; (e) adding to the enriched population of nucleic acids of interest genomic DNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of genomic DNA sequences and the enriched nucleic acid mixture, and (f) sequencing the genomic DNA sequences and cDNA sequences in the new mixture.

In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (e) mixing said enriched genomic DNA of interest and said enriched cDNA of interest to obtain a population of nucleic acids of interest; (f) adding to the population of nucleic acids of interest genomic DNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of genomic DNA sequences and the enriched nucleic acid mixture, and (f) sequencing the genomic DNA sequences and cDNA sequences in the new mixture.

In some embodiments, cDNA sequences obtained from the same test sample can be added to the enriched nucleic acid mixture. The addition of cDNA sequences allows, for example, both broad sequencing (or analyzing) at the transcriptome-wide level and deep sequencing of the nucleic acids of interest. In some embodiments, the desired ratio of cDNA sequences to the nucleic acid mixture is about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000.

Thus, for example, In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) contacting a mixture of nucleic acids with a set of probes under a condition sufficient for hybridization of said nucleic acids to said probes, wherein said probes are complementary to nucleic acids of interest present in said mixture of nucleic acids, and wherein the mixture of nucleic acids comprise genomic DNA sequences and cDNA sequences obtained from the test sample; (b) separating nucleic acids hybridized to said probes from those not hybridized; thereby obtaining an enriched population of nucleic acids of interest; (c) adding to the enriched population of nucleic acids of interest cDNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of cDNA sequences and the enriched nucleic acid mixture, and (d) sequencing the genomic DNA sequences and cDNA sequences in the new mixture. In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) providing a mixture of nucleic acids comprising genomic DNA sequences and cDNA sequences obtained from the test sample; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; (d) adding to the enriched population of nucleic acids of interest cDNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of cDNA sequences and the enriched nucleic acid mixture, and (e) sequencing the genomic DNA sequences and cDNA sequences in the new mixture. In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) mixing a genomic DNA library generated from the test sample and a cDNA library generated from the test sample to provide a mixture of nucleic acids; (b) contacting the mixture of nucleic acids with a set of probes under a condition sufficient for hybridization of said nucleic acids to said probes, wherein said probes are complementary to nucleic acids of interest present in said mixture of nucleic acids; (c) separating nucleic acids hybridized to said probes from those not hybridized; thereby obtaining an enriched population of nucleic acids of interest; (d) adding to the enriched population of nucleic acids of interest cDNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of cDNA sequences the enriched nucleic acid mixture, and (e) sequencing the genomic DNA sequences and cDNA sequences in the new mixture. In some embodiments, the genomic DNA library and the cDNA library are mixed before the enrichment at a predetermined ratio (for example at a ratio of about 10:1, 5:1, 2:1, 1:1, 1:0, 0:1, 1:2, 1:5, 1:10).

In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) reverse transcribing the RNA in the test sample into cDNA; (b) generating a DNA library comprising genomic DNA sequences and cDNA sequences to provide a mixture of nucleic acids; (c) contacting the mixture of nucleic acids with a set of probes under a condition sufficient for hybridization of said nucleic acids to said probes, wherein said probes are complementary to nucleic acids of interest present in said mixture of nucleic acids; (d) separating nucleic acids hybridized to said probes from those not hybridized; thereby obtaining an enriched population of nucleic acids of interest; (e) adding to the enriched population of nucleic acids of interest cDNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of cDNA sequences and the enriched nucleic acid mixture, and (f) sequencing the genomic DNA sequences and cDNA sequences in the new mixture.

In some embodiments, there is provided a method of characterizing nucleic acid (such as obtaining a nucleic acid profile of genomic DNA and RNA and/or simultaneously detecting genetic variations and variations in a RNA transcript) in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (e) mixing said enriched genomic DNA of interest and said enriched cDNA of interest to obtain a population of nucleic acids of interest; (f) adding to the population of nucleic acids of interest cDNA sequences from the test sample to obtain a predetermined ratio (for example at weight ratio of about any of 100,000:1, 10,000:1, 1,000:1, 100:1, 10:1, 5:1, 2:1, 1:1, 1:2, 1:5, 1:10, 1:100, 1:200, 1:300, 1:400, 1:500, 1:600, 1:700, 1:800, 1:900, 1:1,000, 1:10,000, or 1:100,000) of cDNA sequences and the enriched nucleic acid mixture, and (f) sequencing the genomic DNA sequences and cDNA sequences in the new mixture.

In some embodiments, the nucleic acid further comprises genomic DNA and/or cDNA sequences obtained from the control sample. These sequences in the control sample are indexed differently but otherwise processed in the same manner as the test sample. In some embodiments, the control sample is from the same individual. In some embodiments, the control sample is from a different individual.

### Providing a Mixture of Nucleic Acids

The methods of the present application in some embodiments comprise providing a mixture of nucleic acids comprising genomic DNA sequences and cDNA sequences obtained from the same sample, for example a human sample. In some embodiments, the sample is a tissue sample or nucleic acids extracted from a tissue sample. In some embodiments, the sample is a cell sample (for example a CTC sample) or nucleic acids extracted from a cell sample. In some embodiments, the sample is a single cell or nucleic acids extracted from a single cell. In some embodiments, the sample is a tumor sample or nucleic acids extracted from a tumor sample. In some embodiments, the sample is a biopsy sample or nucleic acids extracted from the biopsy sample. In some embodiments, the sample is a Formaldehyde Fixed-Paraffin Embedded (FFPE) sample or nucleic acids extracted from the FFPE sample.

The present application also encompasses any of the nucleic acid mixtures described herein. The nucleic acid mixture described herein can be obtained, for example, by preparing a genomic DNA library and a cDNA library from the test sample separately and then mixing these two libraries together, e.g., at a predetermined ratio.

Genomic DNA library can be obtained, for example, by fragmenting genomic DNA in the sample into genomic DNA fragments. Methods of fragmenting nucleic acids are well known in the art. Exemplary methods include, but are not limited to, enzymatic digestion such as exo- or endonuclease digestion, chemical cleavage, photocleavage, and mechanical forces such as shearing and combinations of these methods.

To facilitate next generation sequencing, the DNA fragments in some embodiments are ligated to platform-specific oligonucleotide adaptors to yield a sequencing-ready library. In some embodiments, the genomic DNA sequences in the library comprise an index that allows differentiation of the genomic DNA sequences with the cDNA sequences in the same mixture. The index is used to designate the genomic DNA sequences and to be able to report information related only to genomic DNA sequences in the test sample, and not other nucleic acid sequences that may be involved in the same experiment. This allows information obtained during the analyses to be traced back to the genomic DNA sequences, even when the genomic DNA sequences are physically mixed with other sequences (such as cDNA sequences) and not physically separated or distinguishable.

Genomic DNA described herein can have one or more chromosomes. For example, a prokaryotic genomic DNA including one chromosome can be used. Alternatively, a eukaryotic genomic DNA including a plurality of chromosomes can be used in a method disclosed herein. Thus, the methods can be used, for example, to select, amplify or analyze a genomic DNA having n equal to 2 or more, 4 or more, 6 or more, 8 or more, 10 or more, 15 or more, 20 or more, 23 or more, 25 or more, 30 or more, or 35 or more chromosomes, where n is the haploid chromosome number and the diploid chromosome count is 2n. The size of a genomic DNA used in a method of the invention can also be measured according to the number of base pairs or nucleotide length of the chromosome complement. Exemplary size estimates for some of the genomes that are useful in the invention are about 3.1 Gbp (human), 2.7 Gbp (mouse), 2.8 Gbp (rat), 1.7 Gbp (zebrafish), 165 Mbp (fruitfly), 13.5 Mbp (S. cerevisiae), 390 Mbp (fiigu), 278 Mbp (mosquito) or 103 Mbp (C. elegans). Those skilled in the art will recognize that genomes having sizes other than those exemplified above including, for example, smaller or larger genomes, can be used.

cDNA library can be obtained, for example, by reverse transcribing RNA in the sample into cDNA. In some embodiments, the RNA is total RNA in the test sample, which include, for example, mRNA, ribosomal RNA, nuclear RNA, cytoplasmic RNA, capped RNA, and small RNA. In some embodiments, the RNA is a processed RNA sample with ribosomal RNA removed. In some embodiments, the RNA is mRNA. To facilitate next generation sequencing, the cDNA or cDNA fragments in some embodiments are ligated to platform-specific oligonucleotide adaptors to yield a sequencing-ready library. In some embodiments, the cloned cDNA sequences comprise an index that allows differentiation of the genomic DNA sequences with the cDNA sequences in the same mixture.

The Genomic DNA library and the cDNA library can be mixed at a predetermined ratio. In some embodiments, the weight ratio of the genomic DNA library and the cDNA library in the nucleic acid mixture is any of about 100:1, 90:1, 80:1, 70:1, 60:1, 50:1, 40:1, 30:1, 20:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, or 1:100. In some embodiments, the weight ratio of the genomic DNA library and the cDNA library in the nucleic acid mixture is about 10:1, about 5:1, about 2:1, about 1:1, about 1:2, about 1:5, or about 1:10.

In some embodiments, total nucleic acid containing both DNA and RNA in the sample can be used directly to generate a mixture of nucleic acids. A reverse transcription reaction can be carried out with the total nucleic acid, generating a population of cDNA. Alternatively, a population of cDNA can be generated after removal of ribosomal RNA. An index can be added during the reverse transcription process, for example, by using an overhang of the random primer used for the reverse transcription reaction, so that the cDNA sequences generated thereby can be distinguished over the genomic DNA sequences. A single library containing both the genomic DNA sequences and the cDNA sequences can then be generated.

In some embodiments, the Genomic DNA and cDNA from the test sample are separately enriched and then mixed together to provide a single mixture of enriched genomic DNA and cDNAs.

### Enriching Nucleic Acids of Interest

The methods described herein in some embodiments comprise enrichment for nucleic acids of interest. The methods generally comprise contacting a mixture of nucleic acids (or genomic DNA library or cDNA library described herein) with a set of probes under a condition sufficient for hybridization of said nucleic acids to said probes, wherein the probes are complementary to nucleic acids of interest present in the mixture. The enrichment methods described herein reduce the complexity of the nucleic acid sequences to be analyzed and allow the nucleic acids of interest to be better represented in the pool.

Thus, in some embodiments, there is provided a method of obtaining an enriched population of nucleic acids of interest in a test sample, comprising: (a) contacting a mixture of nucleic acids with a set of probes under a condition sufficient for hybridization of said nucleic acids to said probes, wherein said probes are complementary to nucleic acids of interest collectively present in the mixture of nucleic acids, wherein the nucleic acid mixture comprises genomic DNA sequences and cDNA sequences obtained from the test sample; and (b) separating nucleic acids hybridized to said probes from those not hybridized; thereby obtaining an enriched population of nucleic acids of interest.

In some embodiments, there is provided a method of obtaining an enriched population of nucleic acids of interest in a test sample, comprising: (a) contacting a genomic DNA library generated from the test sample with a first set of probes under a condition sufficient for hybridization of said genomic DNA to said first set of probes, wherein said first set of probes are complementary to nucleic acids of interest present in said genomic DNA library; (b) separating genomic DNA hybridized to said first set of probes from those not hybridized; thereby obtaining an enriched population of genomic DNA of interest; (c) contacting a cDNA library generated from the test sample with a second set of probes under a condition sufficient for hybridization of said cDNA to said second set of probes, wherein said second set of probes are complementary to cDNA of interest present in said cDNA library; (d) separating cDNA hybridized to said second set of probes from those not hybridized; thereby obtaining an enriched population of cDNA of interest; (e) mixing said enriched genomic DNA of interest and said enriched cDNA of interest to obtain a population of nucleic acids of interest

In some embodiments, the method comprises denaturing the nucleic acid mixture (or genomic DNA or cDNA as described herein) prior to contacting the set of the probes with the mixture. In some embodiments, the method comprises denaturing the nucleic acid mixture (or genomic DNA or cDNA as described herein) after contacting the probes with the mixture. The mixture is then subject to an annealing condition that allows the probes to hybridize to the enriched population of nucleic acids of interest.

In some embodiments the nucleic acids of interest comprise one or more desired regions where oncogenes are located. In some embodiments, the nucleic acids of interest comprise one or more desired regions where tumor suppressors are located. In some embodiments, the nucleic acids of interest comprise one or more desired regions where tyrosine kinases are located. In some embodiments, the nucleic acids of interest comprise one or more desired regions where phosphatases are located. In some embodiments, the nucleic acids of interest comprise one or more desired regions where vascular genes are located. In some embodiments, the nucleic acids of interest comprise one or more desired regions where genetic mutations are located.

In some embodiments, the nucleic acids of interest comprise a single nucleotide variation that is indicative of a disease. In some embodiments, the nucleic acids of interest correspond to gene transcripts that are differentially expressed in a disease sample. In some embodiments, the nucleic acids of interest reflect translocation events in a disease sample. In some embodiments, the nucleic acids of interest correspond to nucleic acids that are subject to copy number variation in a disease sample. In some embodiments, the nucleic acids of interest comprise nucleic acids collectively have more than one characteristics described herein. For example, in some embodiments, the nucleic acids of interest comprise at least one nucleic acid that harbors a single nucleotide variation and at least one nucleic acid that corresponds to a gene transcript that is differentially expressed. In some embodiments, the nucleic acids of interest comprise at least one nucleic acid that reflects a translocation event and at least one nucleic acid that involves copy number variation. In some embodiments, the nucleic acids of interest include, but are not limited to, unique sequences of a genome, genes within a genome, coding regions, exons, introns, intergenic regions, intron/exon junctions, differentially expressed gene transcripts, translocation sites, and the like.

The number of probes may be selected based on the complexity of the sample material and the sequence length desired to be sequenced. The methods described herein may be done using a single probe or a plurality (i.e., a mixture of at least 2, at least 5, at least 10, at least 50, at least 100, at least 500, at least 1000, at least 10,000, at least 100,000, or more) of different probes. These probes can be used to enrich for a plurality (i.e., at least 2, at least 5, at least 10, at least 50, at least 100, at least 500, at least 1000, at least 10,000, at least 100,000, or more) different regions on the nucleic acid sequence.

The set of probes employed in the methods described herein are selected based on the desired nucleic acids of interest. Enrichment of nucleic acids of interest using the methods of the invention in some embodiments entails designing the probes complementary to the predetermined population of these sequences and using them as affinity binders to separate the nucleic acids of interest from undesired sequences within the nucleic acid mixture.

Probes complementary to a predetermined portion of nucleic acids can be designed using nucleic acid sequence information available from a variety of sources and methods well known in the art. For example, nucleic acid sequences, including genomic sequences, can be obtained from any of a variety of sources well known to those skilled in the art. Such sources include for example, user derived, public or private databases, subscription sources and on-line public or private sources. For example, exemplary public databases for obtaining genomic and gene sequences include, for example, UCSC human genome database, dbEST-human, UniGene-human, gb-new-EST, Genbank, Gb_pat, Gb_htgs, Refseq, Derwent Geneseq and Raw Reeds Databases. The nucleic acid sequence information additionally can be generated by a user and used directly or stored, for example, in a local database. Various other sources well known to those skilled in the art for genomic and transcriptome information also exist and can similarly be used for generating the probes.

The probes used in the methods described herein can be of any length, including, but not limited to, about 10 to about 50, about 50 to about 100, about 100 to about 120, about 120 to about 140, about 140 to about 160, about 160 to about 180, about 180 to about 200, about 200 to about 300, about 300 to about 400, or about 400 to about 500 nucleotides long. In some embodiments, the probes are about 100, about 105, about 110, about 115, about 120, about 125, about 130, about 135, about 140, about 145, or about 150 nucleotides long. The probes in some embodiments are provided in excess to the nucleic acids to be enriched. For example, in some embodiments, the probes are at least about any of 1, 2, 5, 10, 10², 10³, 10⁴, or more times the amount of the nucleic acids to be enriched. In some embodiments, the probes are no more than about 10, 10², 10³, or 10⁴ times the amount of the nucleic acids to be enriched. In some embodiments, a molar excess (e.g., at least about any of 2×, 5×, 10×, 15×, 20×, 30×, 40×, 50×, 60×, 70×, 80×, 90×, 100×, or 1000×, or more) of probes compared to the nucleic acid of interest is used.

In some embodiments, at least one of the probes is complementary to a nucleic acid of interest present in a genomic DNA sequence and a nucleic acid of interest present in a cDNA sequence. For example, in some of the embodiments, the probe is complementary to an exon of a gene that can be found both on the genomic DNA sequence and on the cDNA sequence. In some embodiments, the probes are single stranded. In some embodiments, the probes are double stranded, thereby comprising sequences having complementarity to both strands of a nucleic acid of interest. In some embodiments, the probes comprise sequences complementary to regions such as oncogenes, tumor suppressors, kinases, phosphatases, cell cycle genes, growth factor genes, receptor genes, and/or vascular genes. In some embodiments, the probes comprise the Elim RightOn™ 1000 cancer gene panel.

The contacting step can be performed in a solution-phase process in the absence of solid supports. Alternatively, the contacting step can be performed with immobilized sample nucleic acids or with immobilized probes. The mixture of nucleic acids is subject to denaturation prior to contacting with the probe or after the addition of the probes in order to allow hybridization of the probes to the nucleic acids.

The probes described herein are allowed to contact with the mixture of nucleic acids described herein, under a condition that is sufficient for hybridization of the nucleic acids to the probes. Conditions for hybridization in the present invention are generally high stringency conditions as known in the art, although different stringency conditions can be used. Stringency conditions have been described, for example, in Sambrook et al, Molecular Cloning: A Laboratory Manual, 3d ed. (2001) or in Ausubel et al, Current Protocols in Molecular Biology (1998). High stringency conditions favor increased fidelity in hybridization, whereas reduced stringency permit lower fidelity. Stringent conditions are sequence-dependent and will be different in different circumstances. Longer sequences hybridize specifically at higher temperatures. An extensive guide to the hybridization of nucleic acids is found in Tijssen, “Overview of principles of hybridization and the strategy of nucleic acid assays” in Techniques in Biochemistry and Molecular Biology 8212; Hybridization with Nucleic Acid Probes (1993). Generally, stringent conditions are selected to be about 5-10 C.° lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (i.e., as the target sequences are present in excess, at Tm, 50% of the probes are occupied at equilibrium). Stringent conditions may also be achieved with the addition of helix-destabilizing agents such as formamide. Stringency can be controlled by altering a step parameter that is a thermodynamic variable such as temperature or concentrations of formamide, salt, chaotropic salt, pH, and/or organic solvent. These parameters may also be used to control non-specific binding, as is generally outlined in U.S. Pat. No. 5,681,697. Thus it may be desirable to perform certain steps at higher stringency conditions to reduce non-specific binding.

In some embodiments, the probes comprise a tag that allows the probes and nucleic acids hybridized thereto to be recognized and separated. In certain cases, the tag specifically binds to a ligand thereby facilitating the separation. Exemplary pairs of tag/ligand include, but are not limited to, antibody/antigen, antigen/antibody, avidin/biotin, biotin/avidin, streptavidin/biotin, biotin/streptavidin, glutathione/GST, GST/glutathione, maltose binding protein/amylose, amylose/maltose binding protein, cellulose binding protein/cellulose, cellulose/cellulose binding protein, etc. The ligand recognizing the tag can be coupled (directly or indirectly) to a supporting material, which in turn provides a physical or chemical means for separation.

In some embodiments, the probes are attached to a solid support (directly or via a tag) prior to or after being in contact with the mixture of nucleic acids. Nucleic acids unhybridized to the probes can then be separated away by washing, and those hybridize to the probes can then be recovered by an elution step.

Suitable solid supports include, but are not limited to, plates, tubes, bottle, flasks, beads, magnetic beads, magnetic sheets, porous matrices, or any solid surface and the like. Physical separation can be effected, for example, by filtration, isolation, magnetic field, centrifugation, washing, etc.

In some embodiments, the solid support is a bead, a membrane, a cartridge, a filter, a microtiter plate, a test tube, solid powder, a cast or extrusion molded module, a mesh, a fiber, a magnetic particle composite, or any other solid materials. The solid support may be coated with a substance such as polyethylene, polypropylene, poly(4-methulbutene), polystyrene, polyacrylate, polyethylene terephthalate, rayon, nylon, poly(vinyl butyrate), polyvinylidene difluoride (PCDF), silicones, polyformaldehyde, cellulose, cellulose acetate, nitrocellulose, and the like. In some embodiments, the solid support may be coated with a ligand or impregnated with the ligand.

Other solid support that can be used in the methods described herein include, but are not limited to, gelatin, glass, sepharose macrobeads, dextran microcarriers such as CYTODES® (Pharmacia, Uppsala, Sweden). Also contemplated are polysaccharide such as agarose, alginate, carrageenan, chitin, cellulose, dextran or starch, polyacrylamide, polystyrene, polyacrolein, polyvinyl alcohol, polymethylacrylate, perfluorocarbon, inorganic compounds such as silica, glass, kieselquhr, alumina, iron oxide or other metal oxides, or copolymers consisting of any combination of two or more naturally occurring polymers, synthetic polymers or inorganic compounds. In some embodiments, the solid support is a column (such as a Sepharose column).

The probes can be attached to the solid support via a number of methods known in the art. Such methods include, for example, attachment by direct chemical synthesis onto the solid support, chemical attachments, photochemical attachment, thermal attachment, enzymatic attachment, and/or absorption. In some embodiments, the probes are attached to a solid support covalently. In some embodiments, the probes are attached to the solid support via a covalent bond. In some embodiments, the probes are attached to the solid support non-covalently, for example via ligand/tag interactions.

The level of complexity reduction obtained by the enrichment method may enable reduction of 10%, 20%, 30%, 40%, 50%, 60%, 70%, 80%, 90%, 95%, 99%, 99.5%, 99.9%, 99.99%, 99.999%, or more of the complexity of the initial nucleic acid pool, or may involve selection of only a few percent of the nucleic acids, or even a few thousand base pairs. For example, the complexity of the nucleic acids may be reduced from 3 billion base pairs to 10 million base pairs or less, depending on the size of the initial genome and transcriptome and the level of reduction required. Using this method, highly repetitive DNA sequences which comprise, for example 40% of the human genomic DNA, can be removed quickly and efficiently from a complex population.

In some embodiments, the method further comprises amplifying the nucleic acids of interest prior to the analyses, for example by PCR. Such amplification can be carried out, for example, before or after the nucleic acids of interest are eluted from the solid support as described above.

### Analyzing Nucleic Acids

The nucleic acids mixture comprising genomic DNA sequences and cDNA sequences described herein can be further subject to analysis. The analysis can be carried out directly on the nucleic acid mixture, or it is carried out on an enriched population of nucleic acids of interest following the enrichment methods described herein.

The analysis can include, but not limited to, nucleic acid sequencing, mutation analysis, determination of polymorphism, etc. The methods described herein are particularly useful for identifying mutations in a nucleic acid sample, predicting responsiveness of an individual to a drug; predicting pharmacokinetics of drug in an individual, predicting therapeutic outcome of a treatment in an individual. The methods can also be useful for genetic testing such as genetic testing for prenatal screening.

The nucleic acids can be analyzed by any analysis methods, including, but not limited to, DNA sequencing (using Sanger, pyrosequencing or the sequencing systems of Roche/454, Helicos, Illumina/Solexa, and ABI (SOLID)), Life Technology (Ion Torrent), a polymerase chain reaction assay, a bead array assay, a primer extension assay, an enzyme mismatch cleavage assay, a branched hybridization assay, a NASBA assay, a molecular beacon assay, a cycling probe assay, a ligase chain reaction assay, an invasive cleavage structure assay, an ARMS assay, or a sandwich hybridization assay, for example. The nucleic acid molecules can be sequenced or analyzed for the presence of SNPs or other differences relative to a reference sequence.

In some embodiments, the nucleic acids generated by the methods described herein can be used for NP haplotyping of a chromosomal region that contains two or more SNPS, for enriching for DNA sequences for paired-end sequencing methods, for generating target fragments for long-read sequences, isolating inversion, deletion, and translocation breakpoints, for sequencing entire gene regions (exons and introns) to uncover mutations causing aberrant splicing or regulation.

Polymorphisms, such as single nucleotide polymorphism (“SNP”) are essentially randomly distributed throughout the genome. A polymorphism may be an insertion, deletion, duplication, or rearrangement of any length of a sequence, including single nucleotide deletions, insertions, or base change. The polymorphism may be naturally occurring, or it may be associated with variant phenotypes. The use of the methods described herein, for example through the enrichment of the sequences of interest, allows substantially reproducible access to substantially similar reduced-complexity subpopulations in different individuals in a population or even in different samples from a single individual. Because polymorphisms are essentially randomly distributed throughout the genome, a number of polymorphic sequences will be present in the reduced-complexity population of nucleic acid sequences. Such reduced-complexity subpopulation can be analyzed to either identify polymorphisms or to determine the genotype of polymorphic loci within that sub-population.

The methods described herein can also be useful, for example, in the field of pharmacogenomics, which seeks to correlate the knowledge of specific alleles of polymorphic loci with the way in which individuals in a population respond to particular drug. A broad estimate is that, for every drug, between 10% and 40% of individuals do not respond optimally. In order to create a response profile for a given drug, the genotype with regard to polymorphic loci of those individuals receiving the drug must be correlated with the therapeutic outcome of the drug. This is frequently performed with analysis of a large number of polymorphic loci. Once a genetic drug response profile has been estimated by analysis of polymorphic loci in a population, a clinical patient's genotype with respect to those loci related to responses to particular drugs must be determined. Therefore, the ability to identify the sequence of a large number of polymorphic loci in a large number of individuals is important for both establishment of a drug response profile and for identification of an individual's genotype for clinical applications.

The nucleic acids generated using the methods described herein (such as single stranded nucleic acids comprising adaptor(s) and nucleic acids enriched by probes) are subjected to sequencing analysis using the Illumina sequencing method. The Illumina sequencing method includes bridge amplification technology, in which primers bound to a solid phase are used in the extension and amplification of solution phase single stranded nucleic acid acids prior to SBS. (See, e.g., Mercier, et al. (2005) “Solid Phase DNA Amplification: A Brownian Dynamics Study of Crowding Effects.” Biophysical Journal 89: 32-42; Bing, et al. (1996) “Bridge Amplification: A Solid Phase PCR System for the Amplification and Detection of Allelic Differences in Single Copy Genes.” Proceedings of the Seventh International Symposium on Human Identification, Promega Corporation Madison, Wis.)

Illumina sequencing technology entails preparing single stranded nucleic acids flanked with paired-end adapter sequences. Each of the paired-end adapters contains a unique primer hybridization sequence. The nucleic acids are distributed on to a flow cell surface that is coated with single stranded oligonucleotides that correspond to the primer hybridization sequences present on the adapters flanking the single stranded nucleic acids. The single stranded, adapter-ligated nucleic acids are bound to the surface of the flow cell and exposed to reagents for polymerase-based extension. Priming occurs as the free/distal end of a ligated fragment “bridges” to a complementary oligonucleotide on the surface, and during the annealing step, the extension product from one bound primer forms a second bridge strand to the other bound primer. Repeated denaturation and extension results in localized amplification of single molecules in millions of unique locations, creating clonal “clusters” across the flow cell surface.

The flow cell is then placed in a fluidics cassette within a sequencing module, where primers, DNA polymerase, and fluorescently-labeled, reversibly terminated nucleotides, e.g., A, C, G, and T, are added to permit the incorporation of a single nucleotide into each clonal DNA in each cluster. Each incorporation step is followed by the high-resolution imaging of the entire flow cell to identify the nucleotides that were incorporated at each cluster location on the flow cell. After the imaging step, a chemical step is performed to deblock the 3′ ends of the incorporated nucleotides to permit the subsequent incorporation of another nucleotide. Iterative cycles are performed to generate a series of images each representing a single base extension at a specific cluster. This system typically produces sequence reads of up to 20-50 nucleotides. Further details regarding this sequencing system are discussed in, e.g., Bennett, et al. (2005) “Toward the 1,000 dollars human genome.” Pharmacogenomics 6: 373-382; Bennett, S. (2004) “Solexa Ltd.” Pharmacogenomics 5: 433-438; and Bentley, D. R. (2006) “Whole genome re-sequencing.” Curr Opin Genet Dev 16: 545-52.

The first stage in preparing template for the Illumina system is DNA fragmentation, such as by sound energy fragmentation (Covaris).

The methods provided herein can be readily adapted for use with the Illumina platform. Specifically, the adaptor sequences described herein are ideally suited for the purpose of the Illumina sequencing methods.

In some embodiments, the sequencing may be carried out with multiple test samples (and control samples) simultaneously by multiplex sequencing on a high throughput instrument. This can be accomplished, for example, by using individual barcode sequences for each sample so that they can be differentiated during the data analyses.

In some embodiments, the nucleic acids generated by the methods described herein are analyzed using single-molecule real-time sequencing. Single molecule real-time sequencing (SMRT) is another massively parallel sequencing technology that can be used to sequence circularized single stranded nucleic acids in a high-throughput manner. Developed and commercialized by Pacific Biosciences, SMRT technology relies on arrays of multiplexed zero-mode waveguides (ZMWs) in which, e.g., thousands of sequencing reactions can take place simultaneously. The ZMW is a structure that creates an illuminated observation volume that is small enough to observe, e.g., the template-dependent synthesis of a single stranded DNA molecule by a single DNA polymerase (See, e.g., Levene, et al. (2003) “Zero Mode Waveguides for Single Molecule Analysis at High Concentrations,” Science 299: 682-686). When a DNA polymerase incorporates complementary, fluorescently labeled nucleotides into the DNA strand that is being synthesized, the enzyme holds each nucleotide within the detection volume for tens of milliseconds, e.g., orders of magnitude longer than the amount of time it takes an unincorporated nucleotide to diffuse in and out of the detection volume. During this time, the fluorophore emits fluorescent light whose color corresponds to the nucleotide base's identity. Then, as part of the nucleotide incorporation cycle, the polymerase cleaves the bond that previously held the fluorophore in place and the dye diffuses out of the detection volume. Following incorporation, the signal immediately returns to baseline and the process repeats. Additional descriptions of ZMWs and their application in single molecule analyses, such as SMRT sequencing can be found in, e.g., Published U.S. Patent Application No. 2003/0044781, and U.S. Pat. No. 6,917,726, each of which is incorporated herein by reference in its entirety for all purposes. See also, Levene et al. (2003) “Zero Mode Waveguides for single Molecule Analysis at High Concentrations,” Science 299:682-686 and Eid, et al. (2009) “Real-Time DNA Sequencing from Single Polymerase Molecules.” Science 323:133-138.

The nucleic acids generated by the methods described herein can be adapted for use with the SMRT sequencing platform. For example, following synthesis, the single stranded nucleic acids can be circularized using an enzyme that catalyzes the intramolecular ligation of single stranded DNA fragments, e.g., CircLigase™, CircLigase™ II, or ThermoPhage™, and distributed to ZMWs. Alternatively, the™daughter strands can be fragmented prior to circularization. Optionally, sequences of interest can be enriched from a population of fragmented daughter strands, e.g., as described above, prior to circularization.

In some embodiments, the methods further comprise data analyses. For example, de novo sequencing requires assembly of sequencing reads. Whole genome/transcriptome analysis requires comparison with a reference database. Determination of RNA expression levels require algorithms that quantify read counts. Determination of single nucleotide variations requires comparison with reference sequences. Tools and software for data analyses are known in the art.

### Kits and Articles of Manufacture

The present application further provides kits and articles of manufacture for any one of the methods described herein. Any of the components or articles used in the performance of the methods can be usefully packaged into a kit.

For example, the kit can comprise components useful for making a nucleic acid mixture, including reverse transcriptase, primers, adaptors, reagents for library construction, and the like. In some embodiments, the kit comprises or further comprises components useful for enriching the nucleic acids of interest, which include, but not limited to, a set of probes, hybridization reagents, solid support, reagents for amplification, etc. In some embodiments, the kit comprises or further comprises components useful for analyzing the nucleic acids in the mixture (with or without enrichment), including for example reagents for sequencing analyses. In some embodiments, the kit further comprises an instruction for carrying out any one or more of the methods described herein. In some embodiments, the kit further comprises software for data analyses and report.

1. A method of obtaining an enriched population of nucleic acids of interest from a test sample, comprising:
(a) providing a mixture of nucleic acids comprising genomic DNA sequences and cDNA sequences obtained from the test sample;
(b) contacting the mixture of nucleic acids with a set of probes under a condition sufficient for hybridization of said nucleic acids to said probes, wherein said probes are complementary to nucleic acids of interest present in said mixture of nucleic acids; and
(c) separating nucleic acids hybridized to said probes from those not hybridized; thereby obtaining an enriched population of nucleic acids of interest.

2. The method of claim 1, wherein the mixture of nucleic acids is obtained by mixing a genomic DNA library and a cDNA library generated from the test sample.

3. The method of claim 1, wherein the mixture of nucleic acids is obtained by (i) reverse transcribing the RNA in the test sample into cDNA and (ii) generating a DNA library comprising genomic DNA sequences and cDNA sequences to provide a mixture of nucleic acids.

4. The method of claim 1, wherein at least one of the probes is complementary to a nucleic acid of interest present in a genomic DNA sequence and a nucleic acid of interest present in a cDNA sequence.

5. The method of claim 1, wherein the genomic DNA sequence and cDNA sequence are present in the mixture in a predetermined ratio.

6-10. (canceled)

11. The method of claim 1, wherein the probes are attached to a solid support prior to or after being in contact with the mixture of nucleic acids.

12. The method of claim 11, further comprising eluting the probes and nucleic acids of interest hybridized to the probes from the solid support.

13. The method of claim 1, further comprising amplifying said nucleic acids of interest.

14. The method of claim 1, further comprising analyzing the enriched nucleic acids.

15. The method of claim 14, wherein the analysis comprises sequencing the enriched nucleic acids of interest.

16. A method of characterizing nucleic acids in a test sample, comprising: (a) providing a mixture of nucleic acids comprising genomic DNA sequences and cDNA sequences obtained from the test sample; and (b) simultaneously sequencing the genomic DNA sequences and cDNA sequences in the mixture.

17. The method of claim 16, wherein the mixture of nucleic acids is obtained by mixing a genomic DNA library and a cDNA library generated from the test sample.

18. The method of claim 16, wherein the mixture of nucleic acids is obtained by (i) reverse transcribing the RNA in the test sample into cDNA and (ii) generating a DNA library comprising genomic DNA sequences and cDNA sequences to provide a mixture of nucleic acids.

19. The method of claim 16, wherein the characterization comprises determination of variations in the genomic DNA sequence in the test sample.

20-21. (canceled)

22. The method of claim 16, wherein the characterization comprises determination of variations in the RNA transcripts in the test sample.

23. (canceled)

24. The method of claim 16, wherein the method comprises enriching the nucleic acid mixture for nucleic acids of interest prior to the sequencing step.

25. The method of claim 24, wherein the enrichment comprises:
(a) contacting the mixture of nucleic acids with a set of probes under a condition sufficient for hybridization of said nucleic acids to said probes, wherein said probes are complementary to nucleic acids of interest present in said nucleic acid mixture; and
(b) separating nucleic acids that are hybridized to said probes from those not hybridized;
thereby obtaining an enriched population of nucleic acids of interest.

26. The method of claim 24, wherein the method further comprises adding to the enriched population of nucleic acids the initial mixture of nucleic acids prior to the sequencing step.

27. The method of claim 24, wherein the method further comprises adding to the enriched population of nucleic acids genomic DNA sequences prior to the sequencing step.

28. The method of claim 24, wherein the method further comprises adding to the enriched population of nucleic acids cDNA sequences prior to the sequencing step.

29-32. (canceled)
